(19) United States
(12) Patent Application Publication
(10) Pub. No.: US 2018/0306744 A1
(43) Pub. Date: Oct. 25, 2018

(54) ANALYTE MEASUREMENT SYSTEM AND METHOD

(71) Applicant: LifeScan Scotland Limited, Invemess (GB)

(72) Inventors: Michael MALECHA, Muir of Ord (GB); Alan MCNEILAGE, Inverness (GB); Dominic STEELE, Inverness (GB); Anna ZVIKHACHEVSKAYA, Inverness (GB)

(21) Appl. No.: 15/492,226

(22) Filed: Apr. 20, 2017

(51) Int. Cl. G01N 27/327; G01N 33/487; G01N 33/49; G01N 33/543; G01N 27/30

(52) U.S. Cl. CPC G01N 27/327; G01N 33/48707; G01N 27/307; G01N 33/5438; G01N 33/49

(57) ABSTRACT

Systems and methods for determining a concentration of an analyte in a physiological fluid with a biosensor are presented. Current values are measured during application of voltage pulses across electrodes of the biosensor. Different intermediate analyte concentrations are calculated using different subsets of the measured current values and different scaling factors. A first intermediate analyte concentration has a first level of accuracy across a range of analyte concentrations. A second intermediate analyte concentration has a higher level of accuracy in the low range. A third intermediate analyte concentration has a higher level of accuracy in the high range. The concentration of the analyte is determined as a function of the different intermediate analyte concentrations. The second intermediate analyte concentration, the third intermediate analyte concentration or an average, is selected if the first intermediate analyte concentration is in the low range, the high range or in between, respectively.

### TECHNICAL FIELD

This application is generally directed to the field of measurement systems and more specifically to a system and related method for measurement of analytes such as glucose.

### BACKGROUND

Demand continues for low cost, accurate and easy to use diagnostics systems that allow patients and clinicians to measure and monitor a wide variety of analytes and physiological factors. Systems that allow the accurate, safe and cost effective measurement of analytes or physiological blood based properties relating to common health conditions are of particular interest. Examples of such analytes and blood properties include glucose, cholesterol, blood ketones, hematocrit, numerous cardiac health bio markers and blood clotting time. While numerous examples of such diagnostic devices are known, the cost and accuracy of such devices remains of significant concern to patients, insurers and health care professionals alike.

By way of example, the determination of blood analyte concentration is typically performed by means of an episodic measuring device such as a hand-held electronic meter which receives blood samples via enzyme-based test strips and calculates the blood analyte value based on the enzymatic reaction. In some diagnostic devices the test sample viscosity or rate at which a species diffuses are of interest because variations in sample viscosity/diffusion may affect the accuracy of the measurement. For example, in common episodic electrochemical glucose test strip results hematocrit impacts the ability of reactive species to diffuse through the analyte thereby impacting measured response. Information as to the rate of diffusion or viscosity would allow compensation for this effect. In other diagnostic assays the rate at which a species of interest diffuses through the test sample may be indicative of the progression of important integrations between certain reagents and the test sample, such as in certain types of immunoassays. In all of the above cases the ability to simply, accurately and cost effectively measure the rate at which a species of interest diffuses through the test sample would provide an indication of viscosity/diffusion and therefore may be important in calculating the concentration of an analyte.

### BRIEF DESCRIPTION OF THE DRAWINGS

So that the manner in which the features of the invention can be understood, a detailed description of the invention may be had by reference to certain embodiments, some of which are illustrated in the accompanying drawings. It is to be noted, however, that the drawings illustrate only certain embodiments of this invention and are therefore not to be considered limiting of its scope, for the scope of the disclosed subject matter encompasses other embodiments as well. The drawings are not necessarily to scale, emphasis generally being placed upon illustrating the features of certain embodiments of the invention. In the drawings, like numerals are used to indicate like parts throughout the various views.

FIG. 1 depicts an exploded view of a test strip for performing analyte concentration measurements, in accordance with aspects set forth herein;

FIG. 2 depicts a schematic diagram of a test meter, in accordance with aspects set forth herein;

FIG. 3 depicts a redox reaction at an electrode (top) and mass transport by diffusion to the electrode (bottom), in accordance with aspects set forth herein;

FIG. 4 depicts current decays with and without convection, in accordance with aspects set forth herein;

FIG. 5 depicts a schematic representation of the reduction (left) and oxidation (right) of a redox species occurring at an electrode together with their respective current decay curves, in accordance with aspects set forth herein;

FIG. 6 depicts a schematic representation of a current output (solid line) obtained in response to an applied pulsed potential sequence (dotted lines), in accordance with aspects set forth herein;

FIG. 7 depicts a voltage pulse waveform that may be applied to the test strip of FIG. 5 and a current response that may be measured by the test meter of FIG. 6, in accordance with aspects set forth herein;

FIG. 8A depicts current values measured at one of the electrodes of the test strip of FIG. 5 upon application of the voltage pulse waveform of FIG. 7, in accordance with aspects set forth herein;

FIGS. 8B-8E depict subsets of the measured current values of FIG. 8A, in accordance with aspects set forth herein; and

FIGS. 9A-9C depict a method for determining a concentration of an analyte in a physiological fluid, in accordance with aspects set forth herein.

### DETAILED DESCRIPTION

The following Detailed Description should be read with reference to the drawings, in which like elements in different drawings are identically numbered. The drawings, which are not necessarily to scale, depict selected embodiments and are not intended to limit the scope of the invention. The Detailed Description illustrates by way of example, not by way of limitation, the principles of the invention. This description will clearly enable one skilled in the art to make and use the invention, and describes several embodiments, adaptations, variations, alternatives and uses of the invention, including what is presently believed to be the best mode of carrying out the invention.

As used herein, the terms “about” or “approximately” for any numerical values or ranges indicate a suitable dimensional tolerance that allows the part or collection of components to function for its intended purpose as described herein. In addition, as used herein, the terms “patient,” “host,” “user,” and “subject” refer to any human or animal subject and are not intended to limit the systems or methods to human use, although use of the subject techniques in a human patient represents a preferred embodiment.

The present disclosure relates, in part, to analyte measurement systems using expert systems that can select and use multiple intermediate analyte concentration calculations to provide more accurate analyte concentration measurements. Specifically, a multi-pulse waveform may be applied to a biosensor, such as a test strip, to measure the current response. The measured current values may be used to calculate an analyte concentration in multiple different ways (e.g., using multiple different equations), some of which are more accurate under certain circumstances, such as within certain ranges of analyte concentrations, at certain hematocrit levels, etc. Advantageously, the system and method disclosed herein allow for combining the multiple different calculations so that analyte concentration results are more accurate.

By way of explanation, after conducting numerous clinical trials involving large numbers of patients and comparing the results of analyte measurements taken with biosensors (e.g., test strips) with analyte measurements taken with laboratory equipment, new methods have been discovered that demonstrably improve the accuracy of the measurements. As will be explained below, the clinical trials and laboratory testing have been used to derive certain tables of coefficients and scaling factors which may be used in conjunction with an expert system to perform enhanced accuracy analyte concentration measurements.

Generally stated, provided herein, in one embodiment, is a method for determining a concentration of an analyte in a physiological fluid with a biosensor having at least two electrodes. At least three voltage pulses are applied across the two electrodes. The at least three voltage pulses include at least two pulses of opposite polarity. Current values are measured at one of the two electrodes during each of the three voltage pulses. Intermediate analyte concentrations of the analyte are calculated, including a first intermediate analyte concentration using a first subset of the measured current values and a first scaling factor, a second intermediate analyte concentration using a second subset of the measured current values and a second scaling factor, and a third intermediate analyte concentration using a third subset of the measured current values and a third scaling factor.

The first subset and the first scaling factor are selected to provide the calculated first intermediate analyte concentration with a first level of accuracy across a range of analyte concentrations ranging from a low range to a high range. The second subset and the second scaling factor are selected to provide the calculated second intermediate analyte concentration with a second level of accuracy higher than the first level of accuracy in the low range of the analyte concentrations. The third subset and the third scaling factor are selected to provide the calculated third intermediate analyte concentration with a third level of accuracy higher than the first level of accuracy in the high range of the analyte concentrations.

The concentration of the analyte is determined as a function of the first, second and third intermediate analyte concentrations. The second intermediate analyte concentration is selected responsive to the first intermediate analyte concentration being in the low range. The third intermediate analyte concentration is selected responsive to the first intermediate analyte concentration being in the high range. An average (or weighted average) of the second and third intermediate analyte concentrations are selected responsive to the first intermediate analyte concentration being between the low and the high ranges.

In another aspect, a method for determining a concentration of an analyte in a physiological fluid with a biosensor having at least two electrodes is presented. At least three voltage pulses are applied across the two electrodes. The at least three voltage pulses comprising at least two pulses of opposite polarity. Current values are measured at one of the two electrodes during each of the three voltage pulses. Intermediate analyte concentrations of the analyte are calculated including a first intermediate analyte concentration using a first subset of the measured current values and a first scaling factor, a second intermediate analyte concentration using a second subset of the measured current values and a second scaling factor, a third intermediate analyte concentration using a third subset of the measured current values and a third scaling factor, and a fourth intermediate analyte concentration of the analyte using at least one of the current values measured during the third voltage pulse without using a scaling factor.

The first subset and the first scaling factor are selected to provide the calculated first intermediate analyte concentration with a first level of accuracy across a range of analyte concentrations ranging from a low range to a high range. The second subset and the second scaling factor are selected to provide the calculated second intermediate analyte concentration with a second level of accuracy higher than the first level of accuracy in the low range of the analyte concentrations. The third subset and the third scaling factor are selected to provide the calculated third intermediate analyte concentration with a third level of accuracy higher than the first level of accuracy in the high range of the analyte concentrations.

Concentration of the analyte is determined as a function of the first, second and third intermediate analyte concentrations. The first intermediate analyte concentration is selected responsive to a temperature of the physiological fluid being outside a predetermined temperature range. The second intermediate analyte concentration is selected responsive to the first intermediate analyte concentration being in the low range. The third intermediate analyte concentration is selected responsive to the first intermediate analyte concentration being in the high range. An average (or weighted average) of the second and third intermediate analyte concentrations is selected responsive to the first intermediate analyte concentration being between the low and the high ranges. A relative bias value is calculated between the determined analyte concentration and the fourth intermediate analyte concentration. An error is reported responsive to the relative bias value being greater than a predetermined amount.

In a further aspect, a system for determining a concentration of an analyte in a physiological fluid is presented. The system includes a biosensor and a meter for performing various steps. The biosensor has at least two electrodes. At least three voltage pulses are applied across the two electrodes and measure current values. The at least three voltage pulses include at least two pulses of opposite polarity. The current values are measured at one of the two electrodes during each of the three voltage pulses.

Intermediate analyte concentrations of the analyte are calculated including a first intermediate analyte concentration using a first subset of the measured current values and a first scaling factor, a second intermediate analyte concentration using a second subset of the measured current values and a second scaling factor, and a third intermediate analyte concentration using a third subset of the measured current values and a third scaling factor.

The first subset and the first scaling factor are selected to provide the calculated first intermediate analyte concentration with a first level of accuracy across a range of analyte concentrations ranging from a low range to a high range. The second subset and the second scaling factor are selected to provide the calculated second intermediate analyte concentration with a second level of accuracy higher than the first level of accuracy in the low range of the analyte concentrations. The third subset and the third scaling factor are selected to provide the calculated third intermediate analyte concentration with a third level of accuracy higher than the first level of accuracy in the high range of the analyte concentrations.

The concentration of the analyte is determined as a function of the first, second and third intermediate analyte concentrations. The second intermediate analyte concentration is selected responsive to the first intermediate analyte concentration being in the low range. The third intermediate analyte concentration is selected responsive to the first intermediate analyte concentration being in the high range. An average (or weighted average) of the second and third intermediate analyte concentrations is selected responsive to the first intermediate analyte concentration being between the low and the high ranges.

The above embodiments are intended to be merely examples. It will be readily apparent from the following discussion that other embodiments are within the scope of the disclosed subject matter.

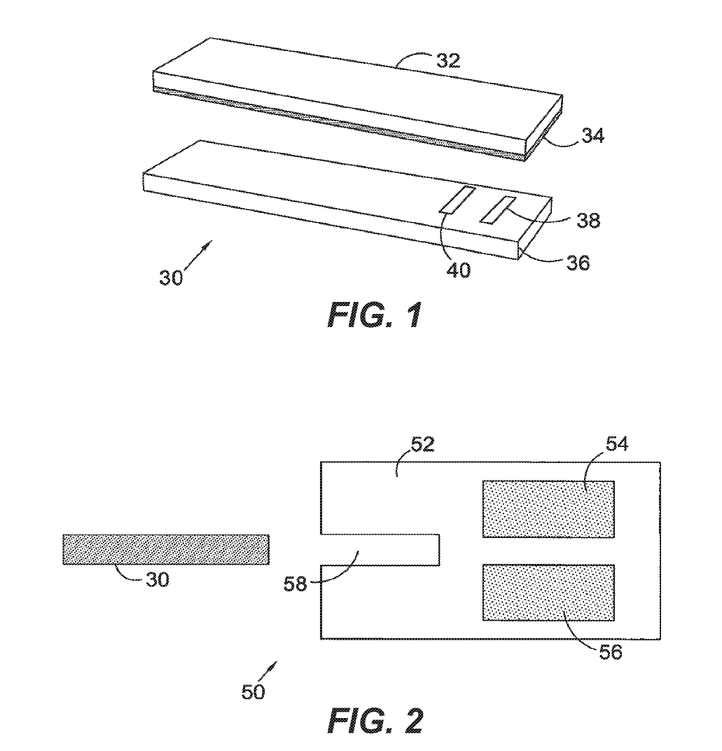

Specific working examples will now be described. Initially, with respect to FIGS. 1-6, a biosensor, test meter, and current measurement technique will be explained.

FIG. 1 depicts an exploded view of a test strip 30 for performing analyte concentration measurements. The test strip 30 has a support insulating layer 36, having at least one pair of electrodes 38 and 40: a working electrode and a counter/reference electrode. A reagent layer (not shown) covers all or part of the support insulating layer. A spacer 34 is sandwiched between the support layer 36 and a carrier substrate 32 (for transporting sample) and forming a sample chamber (not shown) extending around the electrode and where the sample can diffuse.

The electrodes may be made of a material that has a low electrical resistance, such as carbon, gold, platinum or palladium, allowing efficient electrochemistry to take place. The material of the working electrode may be different from the material of the counter/reference electrode. For example, the material of the working electrode should have an electrochemical activity that does not exceed the electrochemical activity of the material of the counter/reference electrode. For example, the working electrode could be made of carbon and a silver or silver chloride reference/counter electrode may be used.

The two electrodes 38 and 40 may be of the same size or of different size. It may be of benefit to regulate by design the degree to which diffusion is defined by radial and planar diffusion. This could be achieved by designing electrodes with high surface to edge ratio to favor planar diffusion or high edge to surface ratio to favor radial diffusion. Another option would be to recess the electrode or border it with walls to limit or prevent radial diffusion. Working and counter/reference electrodes may be coated with the same reagents. These reagents should contain an electrochemically active species capable of undergoing reversible oxidation and reduction. Example species include but are not limited to potassium hexacyanoferrate III, potassium hexacyanoferrate II, ferrocene and ferrocene derivatives, osmium based mediators, gentisic acid and their functionalized derivatives. The reagent layer may also contain ionic salts to support the electrochemistry within the chamber.

The test strip may comprise multiple measuring electrodes allowing different voltage modulation patterns to be applied simultaneously or allowing several diagnostic tests to be carried out simultaneously. For example, the strip may include one or more working electrodes, a counter electrode and a reference electrode. The counter and reference may be the same electrode. The electrodes may optionally be enclosed within a sample chamber, such chamber having at least one aperture suitable for aspirating a sample of blood or other fluid of interest. The fill of the sample chamber may be aided by capillary, wicking, negative driven, electro-wetting or electro-osmotic forces. The reagents disposed on or around the electrodes may contain certain non-active film forming agents in addition to agents that promote the rapid dissolution of the electrochemically active species of interest in to the test sample.

The reagent layer(s) may over coat(s) one or more of the electrodes. In this case, substantially complete dissolution of the layer is required prior to interrogation of the bulk sample solution. Otherwise the layer itself would play a role in defining diffusion-related coefficients. The reagent layer may also be partially soluble over the measurement time. In this case, the rate of dissolution might provide a control measure.

Turning next to FIG. 2, the test strip 30 is controlled using an electronic meter 50 having a test strip port 58 for the insertion of the test strip 30, a voltage control unit 54 configured to apply a voltage across the working and the counter electrodes present on the strip, means of measuring a current generated at the working electrode (not shown), a processor 56 for analyzing the current generated at the working electrode, and a read out display.

The electronic meter 50 determines that the sample is in position via detection of a physical parameter (such as a resistance, capacitance, current, etc. . . . ) reaching a threshold value upon insertion of the strip. The meter 50 may have a voltage control unit 54 that is capable of applying and modulating the potential difference between two electrodes such that the species of interest can be repeatedly oxidized and reduced at the same electrode surface. The pulsed potential waveform may be defined as described below and predetermined by the meter. When the test strip 30 is equipped with multiple electrodes pairs 38 and 40, the control unit 54 can be configured to control each pair separately. In this case each pair 38, 40 may be modulated with a different pulse rate and/or different voltage amplitude. The means for measuring the current is configured to sample current at a frequency equal or greater than 0.2 Hz. The current can be measured at a defined time point or at a peak value. The processor can determine the current rate of change. The meter is configured to perform the methods described below. This can be done under the control of software and/or hardware.

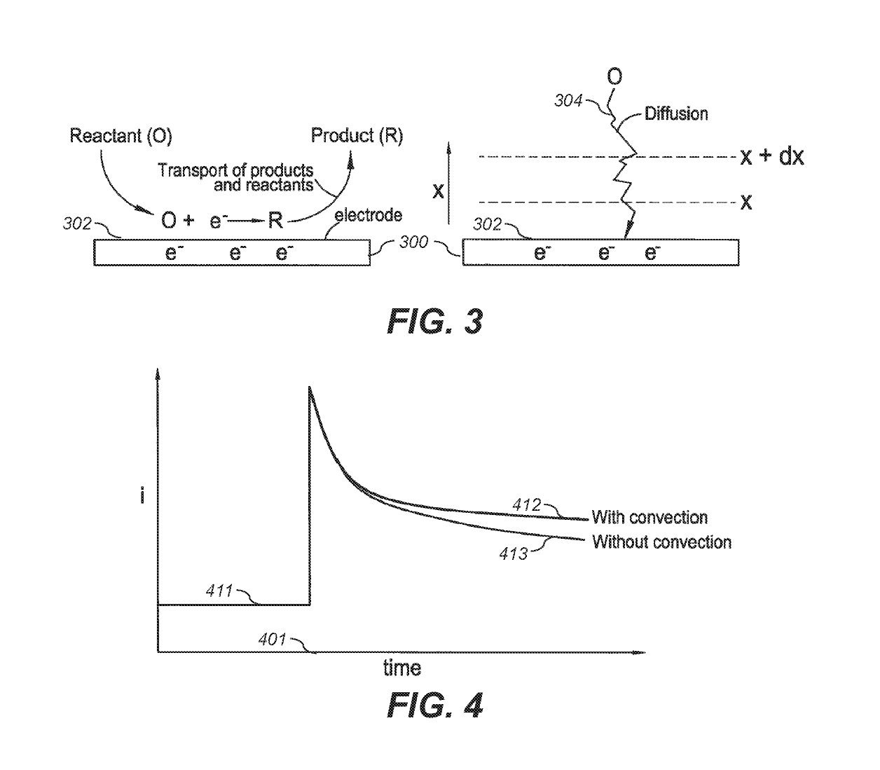

FIG. 3 illustrates the mechanism of oxidation and reduction of a redox species present in a sample and occurring at a surface 302 of an electrode 300. The redox species is represented as an oxidized species O in the oxidized state (i.e., loss of an electron) or a reduced species R in the reduced state (i.e., gain of an electron). The transport of the redox species, from the bulk solution to the surface 302 of the electrode 300, can take place via three principal mechanisms, namely diffusion, migration and convection. If a concentration gradient is present in the sample, molecules may move through diffusion, along a diffusion path 304, from the area of high concentration to the area of low concentration. If an electric field is applied to the sample, charged species will migrate under the influence of the field. In addition, stirring and/or natural thermal motion in the sample triggers the transport of species via convection.

Different types of potentials can be applied to the electrode 300 in order to drive an oxidation or a reduction reaction. The potential at which the redox reaction becomes limited by mass transport is the peak potential. When the potential applied to the electrode 300 is greater than the absolute peak oxidation or reduction potential, the potential is described as an over-potential. An over-potential is a potential of greater than or equal amplitude than that at which the redox reaction at an electrode 300 becomes limited by mass transport. At the over-potential, the theoretical concentration of the analyte being measured is substantially zero at the electrode surface 302 and current diffusion limited. An under-potential is a potential of lesser amplitude than that at which the redox reaction at the electrode 300 becomes limited by mass transport. The under-potential applied to the electrode 300 is less than the absolute peak oxidation or reduction potential (a potential at which current is not solely diffusion limited).

FIG. 4 shows a plot of the expected current output obtained from a pair of electrodes 300 of FIG. 3. Initially, before time value 401, no potential is applied, resulting in a flat line potential 411. Upon application of an over-potential at time value 401, the current rises sharply and decays, a convection profile 412 with convection or without-convection profile 413 without convection. The decay rate is initially very fast and slows down at longer time to reach a “steady-state” current characterized by diffusion. The profile, e.g., the convection profile 412 or the without-convection profile 413, of the current decay can be described by the Cottrell equation where mass transport is driven by diffusion only. Where convection is present, the decay is limited by the increased rate of mass transport of the redox species. The Cottrell equation is given by:

$$i = \frac{n {FAc}_{j}^{0} \sqrt{{D}_{j}}}{\sqrt{π t}} ,$$

where:
i is the current in amperes;
n is the number of electrons to reduce or oxidize one molecule of analyte;
F is the Faraday constant;
cⱼ⁰ is the initial concentration of the reducible analyte in mol/cm³;
Dⱼ is the diffusion coefficient for the species in cm²/s; and
t is the time in seconds.

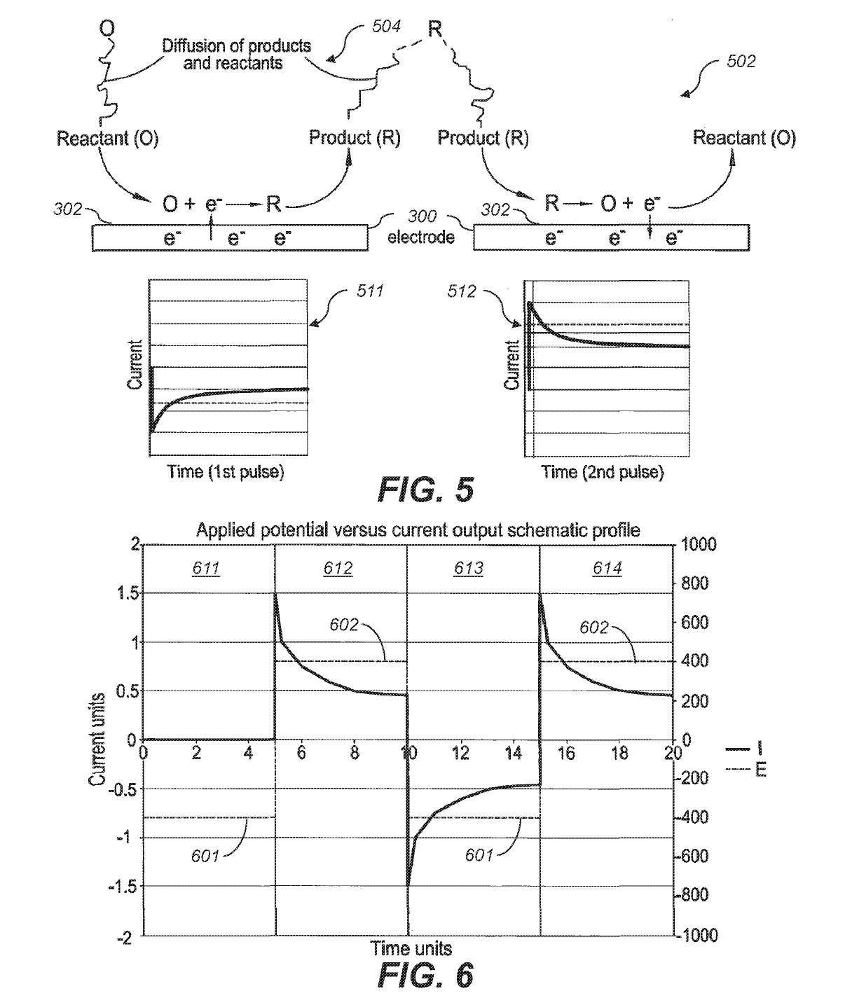

FIG. 5 shows the reduction 501 of the oxidized species O at the surface 302 of the electrode 300, and the oxidation 502 of the reduced species R at the surface 302 of the electrode 300. Successive oxidation and reduction of the redox species is used to determine the rate of mass transport of the species to the electrode 300. Where mass transport is dominated by diffusion, a diffusion-related factor (DRF) of the redox species may be determined. The concentration of the redox species need not be homogenous throughout the solution and the determination can tolerate some degree of convection.

In qualitative terms, during the reduction 501 upon application of an under-potential, the current response is as depicted in graph 511, which shows a peak negative current followed by a current decay. In addition, during the oxidation 502 upon application of an over-voltage, the current response is as depicted in graph 512, which shows a peak positive current followed by a current decay. These current curves are predicted by the Cottrell equation, as noted above.

FIG. 6 is a generalized representation of the input potential E (dotted lines) versus output current I (solid lines). The potential is pulsed between over-potentials 602 and under-potentials 601 for oxidation and reduction. During an initial period 611 a conditioning potential may be applied in order to convert redox species (referred to as mediating species when used as a mediator to measure the concentration of an analyte), to a substantially uniform state (i.e., substantially oxidized or substantially reduced). In such an example the polarity of the applied potential during the initial period 611 could be configured to convert the mediating species to a reduced state. In each of periods 612, 613, and 614, the current is first dominated principally by capacitance. Then, the capacitive element of the current is significantly reduced and the current decay is representative of mediating species being oxidized, i.e., during the period 612 and 614, or reduced, i.e., during the period 613, near the electrode. At the end of each of the periods 612-614, i.e., when the current curves begin to flatten, the current is defined by mediating species diffusing to the electrode through the bulk solution. Therefore, later time points in each of the time periods 612-614 represent mediating species diffusing from greater distance to the electrode.

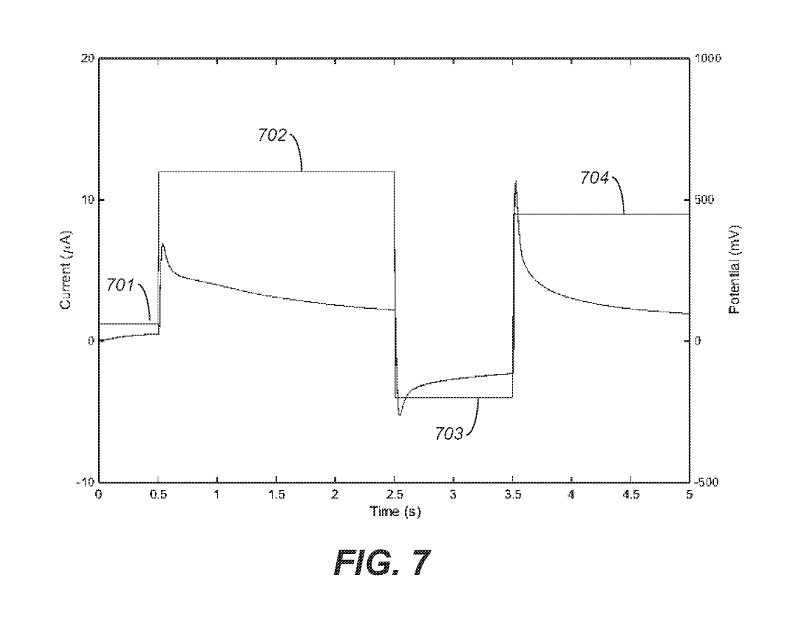

FIG. 7 depicts a voltage pulse waveform (rectangular step waveform) that may be applied to the test strip 30 of FIG. 1 and also depicts a sample current response that may be measured by the test meter 50 of FIG. 2. The voltage pulse of FIG. 7 is specified as set forth in Table 1.

TABLE 1
Specification of Voltage Pulse of FIG. 7

| Pulse | Start Time [s] | End Time [s] | Description |
|---|---|---|---|
| Pulse 701 | 0.0 | 0.5 | Pulse Delay |
| Pulse 702 | 0.5 | 2.5 | Positive |
| Pulse 703 | 2.5 | 3.5 | Negative |
| Pulse 704 | 3.5 | 5.5 | Positive |

By way of explanation and when measuring an analyte concentration, the same voltage pulse will be applied, and different current responses will be measured during each such measurement. The current response shown in FIG. 7 depicts an example measurement, which is provided for ease of understanding. In addition, the positive voltages are the over-potentials described above. Note that this example is used for illustrative purposes only, and numerous other multi-pulse waveforms may be selected with different durations, voltages, etc.

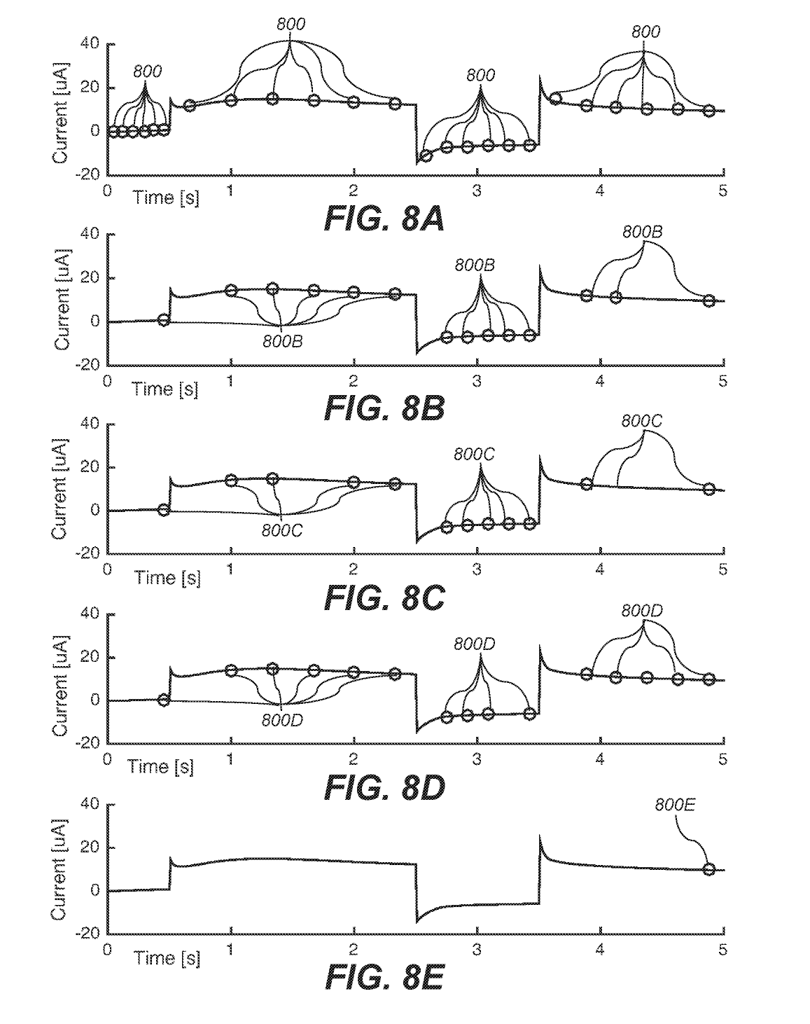

Continuing with the example waveform of FIG. 7, FIG. 8A depicts current value data points 800 measured at one of the electrodes 300 of the test strip 30 of FIG. 5 upon application of the voltage pulse waveform of FIG. 7. In the example of FIG. 8A, a total of eighteen (18) current values have been measured to yield the data points 800.

An equation for calculating an intermediate analyte concentration G is set forth as follows:

$$G = c + {\sum }_{i = 1}^{N} {\sum }_{j = 1}^{N} S {a}_{ij} {x}_{i} {x}_{j}$$

where
G is an intermediate analyte concentration,
N is a number of a subset of the measured current values,
xᵢ, for i=1 to N, are the subset of the measured current values, e.g., at that i-th time period,
aᵢⱼ, for i=1 to N and j=1 to N, are predetermined coefficients,
S is a scaling factor, and
c is a constant.

In other examples, a more general polynomial equation in the variables xᵢ may be used to calculate G, for example including terms such as b_{i,j,n,m}xᵢⁿxⱼ^{m}, where n and m range from zero to 3 (i.e., for a general cubic equation), and b_{i,j,n,m} is a coefficient.

For the examples of FIGS. 8B-8D, the scaling factors may be selected as a ratio of two particular current values selected from the subset xᵢ, as set forth in Table 2. The selection of the particular values of the scaling factors in this example is by way of illustration only, and not by way of limitation. In other examples, different numerators, denominators, or both may be chosen for the scaling factors, and the numerators and/or denominators may be averages or weighted averages of more than one point value.

TABLE 2
Scaling Factors

| Scaling Factor | Numerator | Denominator |
|---|---|---|
| FIG. 8B; S₁ | Pulse 1, point 6 | Pulse 3, point 2 |
| FIG. 8C; S₂ | Pulse 2, point 5 | Pulse 4, point 2 |
| FIG. 8D; S₃ | Pulse 3, point 2 | Pulse 4, point 2 |

FIG. 8B depicts a first subset 800B of the measured current values, in which fourteen (14) of the data points 800 of FIG. 8A have been selected. Following the example of FIG. 8A, an equation as follows may be used to calculate a first intermediate analyte concentration:

$${G}_{1} = {c}_{1} + {\sum }_{i = 1}^{14} {\sum }_{j = 1}^{14} {S}_{1} {a}_{ij}^{1} {x}_{i} {x}_{j}$$

where
G₁ is the first intermediate analyte concentration,
xᵢ, for i=1 to 14, are the subset of the measured current values, e.g., at that i-th time period,
aᵢⱼ¹, for i=1 to 14 and j=1 to 14, are predetermined coefficients,
S₁ is a scaling factor, and
c₁ is a constant.

The constant and coefficients for this calculation are set forth in Table 3, in which each row represents a term which is to be multiplied by a coefficient (or an intercept term which is a constant), and the rows are added to calculate G₁.

TABLE 3
G₁ Calculation

| Term | Coefficient |
|---|---|
| intercept | −6.0 |
| x₁ | −48.7 |
| x₂ | 91.1 |
| x₃ | −380.2 |
| x₄ | 652.9 |
| x₅ | −390.5 |
| x₆ | −8.6 |
| x₇ | −59.3 |
| x₈ | 898.0 |
| x₉ | −361.4 |
| x₁₀ | −700.0 |
| x₁₁ | 159.0 |
| x₁₂ | 395.7 |
| x₁₃ | −765.1 |
| x₁₄ | 503.5 |
| x₁ · x₂ | −152.9 |
| x₁ · x₃ | 829.3 |
| x₁ · x₄ | −1784.3 |
| x₁ · x₅ | 1486.7 |
| x₁ · x₆ | −267.1 |
| x₁ · x₇ | 56.8 |
| x₁ · x₈ | −1950.6 |
| x₁ · x₉ | 2903.0 |
| x₁ · x₁₀ | 26.0 |
| x₁ · x₁₁ | −1179.6 |
| x₁ · x₁₂ | −611.5 |
| x₁ · x₁₃ | 981.3 |
| x₁ · x₁₄ | −609.1 |
| x₂ · x₃ | −201.2 |
| x₂ · x₄ | 717.2 |
| x₂ · x₅ | −996.5 |
| x₂ · x₆ | 382.3 |
| x₂ · x₇ | −23.0 |
| x₂ · x₈ | 769.8 |
| x₂ · x₉ | −1022.4 |
| x₂ · x₁₀ | 155.1 |
| x₂ · x₁₁ | 162.5 |
| x₂ · x₁₂ | 52.6 |
| x₂ · x₁₃ | −59.3 |
| x₂ · x₁₄ | 158.1 |
| x₃ · x₄ | −2240.6 |
| x₃ · x₅ | 3394.8 |
| x₃ · x₆ | −1512.8 |
| x₃ · x₇ | −12.1 |
| x₃ · x₈ | 68.1 |
| x₃ · x₉ | 431.9 |
| x₃ · x₁₀ | −2748.5 |
| x₃ · x₁₁ | 2130.1 |
| x₃ · x₁₂ | 811.5 |
| x₃ · x₁₃ | −1697.7 |
| x₃ · x₁₄ | 438.5 |
| x₄ · x₅ | −7521.8 |
| x₄ · x₆ | 3566.8 |
| x₄ · x₇ | 61.9 |
| x₄ · x₈ | −3519.9 |
| x₄ · x₉ | 1417.6 |
| x₄ · x₁₀ | 6875.0 |
| x₄ · x₁₁ | −4613.0 |
| x₄ · x₁₂ | −4193.2 |
| x₄ · x₁₃ | 7552.5 |
| x₄ · x₁₄ | −2640.0 |
| x₅ · x₆ | −5863.0 |
| x₅ · x₇ | −63.2 |
| x₅ · x₈ | 4800.4 |
| x₅ · x₉ | −253.2 |
| x₅ · x₁₀ | −6971.0 |
| x₅ · x₁₁ | 2128.3 |
| x₅ · x₁₂ | 7294.9 |
| x₅ · x₁₃ | −12853.0 |
| x₅ · x₁₄ | 5108.5 |
| x₆ · x₇ | −27.9 |
| x₆ · x₈ | −1800.3 |
| x₆ · x₉ | −973.5 |
| x₆ · x₁₀ | 2228.4 |
| x₆ · x₁₁ | 786.2 |
| x₆ · x₁₂ | −4329.5 |
| x₆ · x₁₃ | 7735.3 |
| x₆ · x₁₄ | −3498.9 |
| x₇ · x₈ | −15.9 |
| x₇ · x₉ | −559.9 |
| x₇ · x₁₀ | 1758.2 |
| x₇ · x₁₁ | −1288.2 |
| x₇ · x₁₂ | 386.1 |
| x₇ · x₁₃ | −735.8 |
| x₇ · x₁₄ | 435.5 |
| x₈ · x₉ | 395.1 |
| x₈ · x₁₀ | −31254.0 |
| x₈ · x₁₁ | 18570.0 |
| x₈ · x₁₂ | 1862.1 |
| x₈ · x₁₃ | −3048.2 |
| x₈ · x₁₄ | 855.2 |
| x₉ · x₁₀ | 18147.0 |
| x₉ · x₁₁ | −12954.0 |
| x₉ · x₁₂ | 1541.8 |
| x₉ · x₁₃ | −4008.7 |
| x₉ · x₁₄ | 2864.7 |
| x₁₀ · x₁₁ | −29037.0 |
| x₁₀ · x₁₂ | −2425.3 |
| x₁₀ · x₁₃ | 3276.6 |
| x₁₀ · x₁₄ | −70.9 |
| x₁₁ · x₁₂ | −2047.6 |
| x₁₁ · x₁₃ | 5520.8 |
| x₁₁ · x₁₄ | −4418.5 |
| x₁₂ · x₁₃ | −5857.2 |
| x₁₂ · x₁₄ | 3802.5 |
| x₁₃ · x₁₄ | −7560.7 |
| x₁² | 40.4 |
| x₂² | 8.1 |
| x₃² | 398.6 |
| x₄² | 2542.4 |
| x₅² | 5591.8 |
| x₆² | 1767.7 |
| x₇² | 20.4 |
| x₈² | 5932.0 |
| x₉² | −2738.8 |
| x₁₀² | 20488.0 |
| x₁₁² | 12525.0 |
| x₁₂² | 1273.2 |
| x₁₃² | 6185.3 |
| x₁₄² | 2265.9 |

The constant and coefficients may be chosen so that G₁ provides a general analyte concentration that has a wide range of applicability across analyte concentration levels.

FIG. 8C depicts a second subset 800C of the measured current values, in which twelve (12) of the data points 800 of FIG. 8A have been selected. Following the example of FIG. 8A, an equation as follows may be used to a second intermediate analyte concentration:

$${G}_{2} = {c}_{2} + {\sum }_{i = 1}^{12} {\sum }_{j = 1}^{12} {S}_{2} {a}_{ij}^{2} {x}_{i} {x}_{j}$$

where
G₂ is the second intermediate analyte concentration,
xᵢ, for i=1 to 12, are the subset of the measured current values, e.g., at that i-th time period,
aᵢⱼ², for i=1 to 12 and j=1 to 12, are predetermined coefficients,
S₂ is a scaling factor, and
c₂ is a constant.

The constant and coefficients for this calculation are set forth in Table 4, in which each row represents a term which is to be multiplied by a coefficient (or an intercept term which is a constant), and the rows are added to calculate G₂.

TABLE 4
G₂ Calculation

| Term | Coefficient |
|---|---|
| Intercept | −1.5 |
| x₁ | −94.5 |
| x₂ | −4.0 |
| x₃ | 16.3 |
| x₄ | 38.8 |
| x₅ | −122.5 |
| x₆ | 22.1 |
| x₇ | 224.9 |
| x₈ | −413.8 |
| x₉ | 21.7 |
| x₁₀ | 82.6 |
| x₁₁ | −185.1 |
| x₁₂ | 382.9 |
| x₁ · x₂ | 59.3 |
| x₁ · x₃ | −38.9 |
| x₁ · x₄ | 285.6 |
| x₁ · x₅ | −129.6 |
| x₁ · x₆ | 242.5 |
| x₁ · x₇ | −795.0 |
| x₁ · x₈ | 987.1 |
| x₁ · x₉ | 280.8 |
| x₁ · x₁₀ | −741.5 |
| x₁ · x₁₁ | 44.1 |
| x₁ · x₁₂ | −307.0 |
| x₂ · x₃ | −49.7 |
| x₂ · x₄ | 288.4 |
| x₂ · x₅ | −286.2 |
| x₂ · x₆ | −119.0 |
| x₂ · x₇ | 539.2 |
| x₂ · x₈ | −495.3 |
| x₂ · x₉ | −322.4 |
| x₂ · x₁₀ | 370.2 |
| x₂ · x₁₁ | −58.2 |
| x₂ · x₁₂ | 124.6 |
| x₃ · x₄ | −1004.1 |
| x₃ · x₅ | 943.8 |
| x₃ · x₆ | 287.8 |
| x₃ · x₇ | −1074.4 |
| x₃ · x₈ | 892.2 |
| x₃ · x₉ | 434.8 |
| x₃ · x₁₀ | −474.6 |
| x₃ · x₁₁ | 126.0 |
| x₃ · x₁₂ | −326.4 |
| x₄ · x₅ | −3908.7 |
| x₄ · x₆ | −433.1 |
| x₄ · x₇ | 1251.0 |
| x₄ · x₈ | −155.5 |
| x₄ · x₉ | −365.1 |
| x₄ · x₁₀ | −397.0 |
| x₄ · x₁₁ | −350.3 |
| x₄ · x₁₂ | 1087.3 |
| x₅ · x₆ | 149.7 |
| x₅ · x₇ | −1033.0 |
| x₅ · x₈ | 743.5 |
| x₅ · x₉ | −187.6 |
| x₅ · x₁₀ | 333.8 |
| x₅ · x₁₁ | 297.1 |
| x₅ · x₁₂ | −1115.8 |
| x₆ · x₇ | 1388.5 |
| x₆ · x₈ | −1765.8 |
| x₆ · x₉ | 928.3 |
| x₆ · x₁₀ | −287.7 |
| x₆ · x₁₁ | 107.1 |
| x₆ · x₁₂ | 42.5 |
| x₇ · x₈ | −5613.4 |
| x₇ · x₉ | −4149.2 |
| x₇ · x₁₀ | 6837.6 |
| x₇ · x₁₁ | −556.3 |
| x₇ · x₁₂ | 1149.4 |
| x₈ · x₉ | 4271.2 |
| x₈ · x₁₀ | −13078.0 |
| x₈ · x₁₁ | 155.7 |
| x₈ · x₁₂ | −1889.2 |
| x₉ · x₁₀ | 2142.9 |
| x₉ · x₁₁ | 67.6 |
| x₉ · x₁₂ | 684.6 |
| x₁₀ · x₁₁ | 244.6 |
| x₁₀ · x₁₂ | 80.8 |
| x₁₁ · x₁₂ | −119.1 |
| x₁² | 76.0 |
| x₂² | −8.6 |
| x₃² | 146.7 |
| x₄² | 1987.9 |
| x₅² | 1966.7 |
| x₆² | −88.1 |
| x₇² | 702.5 |
| x₈² | 7757.9 |
| x₉² | −1376.1 |
| x₁₀² | 2365.4 |
| x₁₁² | 38.8 |
| x₁₂² | 240.2 |

The constant and coefficients may be chosen so that G₂ provides an analyte concentration that is more accurate at low glucose levels.

FIG. 8D depicts a third subset 800D of the measured current values, in which fifteen (15) of the data points 800 of FIG. 8A have been selected. Following the example of FIG. 8A, an equation as follows may be used to a third intermediate analyte concentration:

$${G}_{3} = {c}_{3} + {\sum }_{i = 1}^{15} {\sum }_{j = 1}^{15} {S}_{3} {a}_{ij}^{3} {x}_{i} {x}_{j}$$

where
G₃ is the third intermediate analyte concentration,
xᵢ, for i=1 to 15, are the subset of the measured current values, e.g., at that i-th time period,
aᵢⱼ³, for i=1 to 15 and j=1 to 15, are predetermined coefficients,
S₃ is a scaling factor, and
c₃ is a constant.

The constant and coefficients for this calculation are set forth in Table 5, in which each row represents a term which is to be multiplied by a coefficient (or an intercept term which is a constant), and the rows are added to calculate G₃.

TABLE 5
Coefficients aᵢⱼ³

| Term | Coefficient |
|---|---|
| intercept | −41.1 |
| x₁ | −66.1 |
| x₂ | −48.9 |
| x₃ | 135.4 |
| x₄ | −75.6 |
| x₅ | −214.9 |
| x₆ | 231.4 |
| x₇ | −3.6 |
| x₈ | −215.8 |
| x₉ | 259.5 |
| x₁₀ | 91.5 |
| x₁₁ | 210.9 |
| x₁₂ | −247.9 |
| x₁₃ | −5.1 |
| x₁₄ | 148.0 |
| x₁₅ | −127.4 |
| x₁ · x₂ | −53.0 |
| x₁ · x₃ | 258.1 |
| x₁ · x₄ | −409.6 |
| x₁ · x₅ | 26.9 |
| x₁ · x₆ | 242.0 |
| x₁ · x₇ | −62.9 |
| x₁ · x₈ | −71.1 |
| x₁ · x₉ | 339.7 |
| x₁ · x₁₀ | −137.5 |
| x₁ · x₁₁ | 29.3 |
| x₁ · x₁₂ | −293.2 |
| x₁ · x₁₃ | −59.9 |
| x₁ · x₁₄ | 2223.9 |
| x₁ · x₁₅ | −1974.8 |
| x₂ · x₃ | 128.3 |
| x₂ · x₄ | −81.2 |
| x₂ · x₅ | −90.7 |
| x₂ · x₆ | 75.1 |
| x₂ · x₇ | −20.8 |
| x₂ · x₈ | 359.9 |
| x₂ · x₉ | −377.0 |
| x₂ · x₁₀ | 28.1 |
| x₂ · x₁₁ | −199.0 |
| x₂ · x₁₂ | 591.7 |
| x₂ · x₁₃ | −131.4 |
| x₂ · x₁₄ | −723.1 |
| x₂ · x₁₅ | 483.5 |
| x₃ · x₄ | 791.2 |
| x₃ · x₅ | −135.2 |
| x₃ · x₆ | −183.9 |
| x₃ · x₇ | 74.8 |
| x₃ · x₈ | −684.3 |
| x₃ · x₉ | 629.5 |
| x₃ · x₁₀ | 0.2 |
| x₃ · x₁₁ | 847.0 |
| x₃ · x₁₂ | −2453.8 |
| x₃ · x₁₃ | 1001.3 |
| x₃ · x₁₄ | 1352.4 |
| x₃ · x₁₅ | −797.5 |
| x₄ · x₅ | 686.1 |
| x₄ · x₆ | 88.2 |
| x₄ · x₇ | −29.7 |
| x₄ · x₈ | 793.7 |
| x₄ · x₉ | −1524.0 |
| x₄ · x₁₀ | 746.0 |
| x₄ · x₁₁ | −1782.9 |
| x₄ · x₁₂ | 5028.4 |
| x₄ · x₁₃ | −3339.8 |
| x₄ · x₁₄ | 30.5 |
| x₄ · x₁₅ | 178.4 |
| x₅ · x₆ | −248.3 |
| x₅ · x₇ | −36.8 |
| x₅ · x₈ | −1324.6 |
| x₅ · x₉ | 3420.1 |
| x₅ · x₁₀ | −2170.4 |
| x₅ · x₁₁ | 2370.6 |
| x₅ · x₁₂ | −7250.1 |
| x₅ · x₁₃ | 6291.2 |
| x₅ · x₁₄ | −1615.0 |
| x₅ · x₁₅ | 97.5 |
| x₆ · x₇ | −43.7 |
| x₆ · x₈ | 1139.1 |
| x₆ · x₉ | −2638.0 |
| x₆ · x₁₀ | 1664.0 |
| x₆ · x₁₁ | −1383.8 |
| x₆ · x₁₂ | 4434.5 |
| x₆ · x₁₃ | −3893.6 |
| x₆ · x₁₄ | 895.1 |
| x₆ · x₁₅ | −84.4 |
| x₇ · x₈ | 27.9 |
| x₇ · x₉ | 358.8 |
| x₇ · x₁₀ | −345.3 |
| x₇ · x₁₁ | 79.2 |
| x₇ · x₁₂ | −98.9 |
| x₇ · x₁₃ | −220.6 |
| x₇ · x₁₄ | 528.9 |
| x₇ · x₁₅ | −200.5 |
| x₈ · x₉ | −7473.5 |
| x₈ · x₁₀ | 1897.2 |
| x₈ · x₁₁ | −184.3 |
| x₈ · x₁₂ | 1123.2 |
| x₈ · x₁₃ | −3015.0 |
| x₈ · x₁₄ | 3898.6 |
| x₈ · x₁₅ | −2219.9 |
| x₉ · x₁₀ | −4043.0 |
| x₉ · x₁₁ | 3078.4 |
| x₉ · x₁₂ | −8051.3 |
| x₉ · x₁₃ | 5227.1 |
| x₉ · x₁₄ | −3754.3 |
| x₉ · x₁₅ | 4235.7 |
| x₁₀ · x₁₁ | −3057.9 |
| x₁₀ · x₁₂ | 7024.9 |
| x₁₀ · x₁₃ | −1757.8 |
| x₁₀ · x₁₄ | −283.5 |
| x₁₀ · x₁₅ | −2362.1 |
| x₁₁ · x₁₂ | −6059.2 |
| x₁₁ · x₁₃ | −292.6 |
| x₁₁ · x₁₄ | 3277.9 |
| x₁₁ · x₁₅ | 152.0 |
| x₁₂ · x₁₃ | 10735.0 |
| x₁₂ · x₁₄ | −14870.0 |
| x₁₂ · x₁₅ | 1783.2 |
| x₁₃ · x₁₄ | 35323.0 |
| x₁₃ · x₁₅ | −12039.0 |
| x₁₄ · x₁₅ | 15532.0 |
| x₁² | −36.5 |
| x₂² | −22.5 |
| x₃² | −285.1 |
| x₄² | −776.6 |
| x₅² | −92.1 |
| x₆² | 162.3 |
| x₇² | −11.7 |
| x₈² | 2654.2 |
| x₉² | 5592.6 |
| x₁₀² | 1363.8 |
| x₁₁² | 1594.0 |
| x₁₂² | 3901.3 |
| x₁₃² | −16819.0 |
| x₁₄² | −19542.0 |
| x₁₅² | −2629.7 |

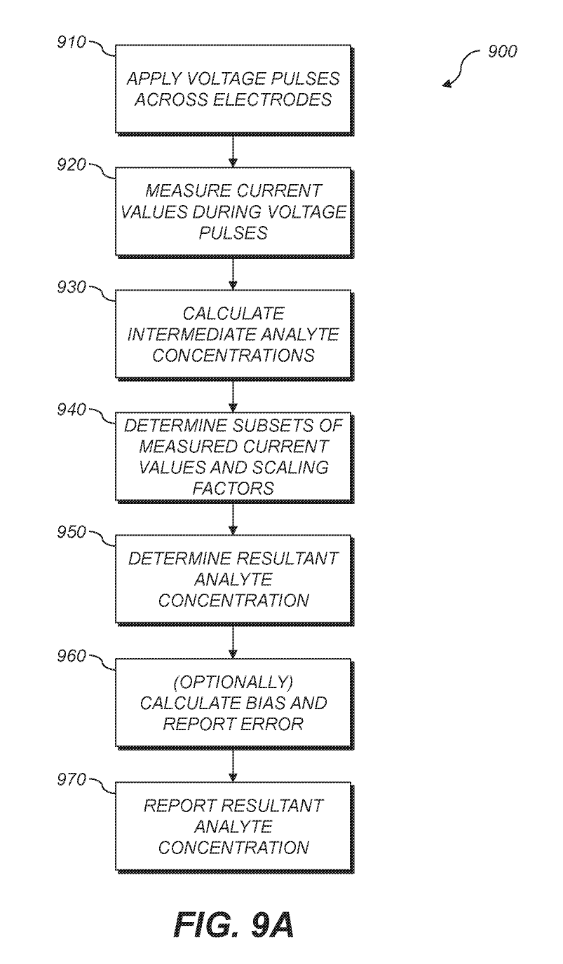

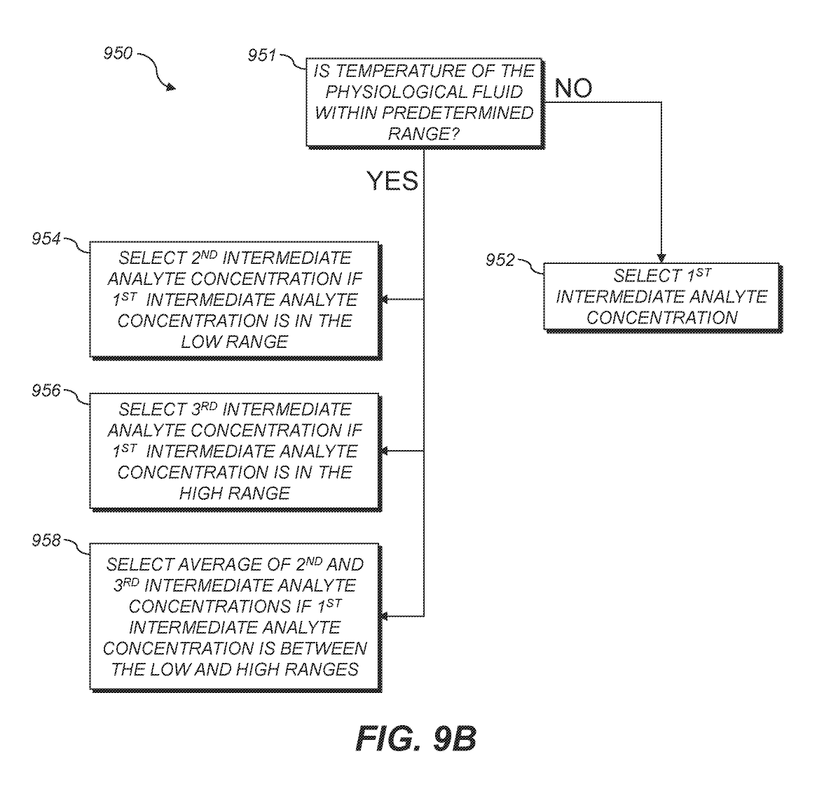

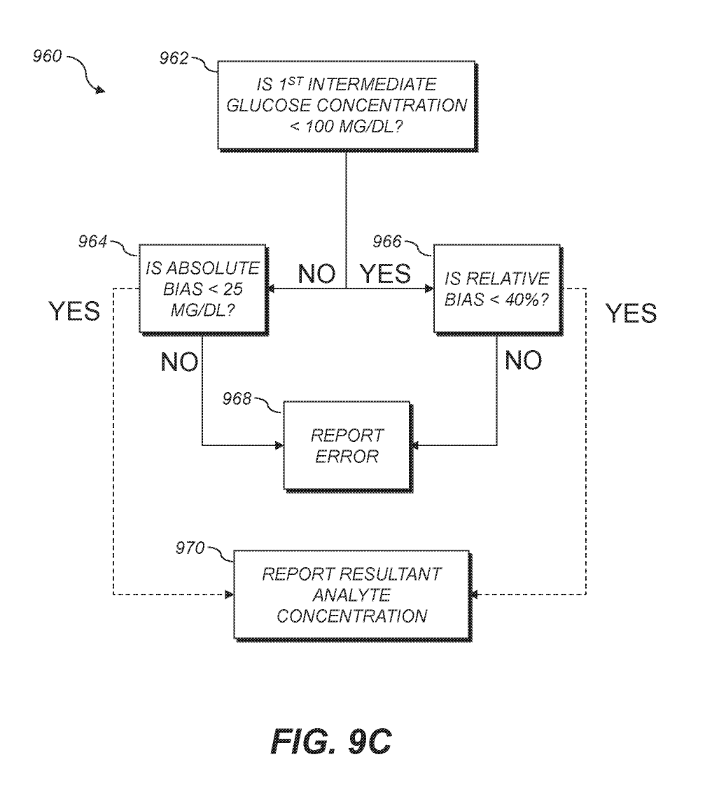

FIG. 8E depicts a fourth subset 800E of the measured current values, in which only one of the data points 800 of FIG. 8A has been selected. In this example, only a single measured current value at the end of the test sequence is used without a scaling factor, which can provide an overall check in conjunction with calculation of a bias value as explained below with respect to FIG. 9C.

In one example, a simple polynomial equation in the single measured current value may be used to calculate a fourth intermediate analyte concentration G₄, as set forth below:

G₄=a+bx+cx²+dx³, where a=−16, b=63, c=1.8, and d=0.003.

FIG. 9A depicts a method 900 for determining a concentration of an analyte in a physiological fluid. For instance, the method 900 is performed on the test meter 50 of FIG. 2 using the test strip 30 of FIG. 1.

In one embodiment, the method 900 at block 910 applies at least three (3) voltage pulses across the two electrodes, which may include the electrodes 38, 40 as described with respect to FIG. 1. In one example, the at least three voltage pulses may include at least two pulses of opposite polarity, such as the voltage pulses depicted in FIG. 7. Next, the method 900 at block 920 measures current values at one of the two electrodes during each of the three voltage pulses. For example, the measurement may take place at the working electrode. Numerous current measurements may be taken during each pulse. In one example, each pulse may be divided into six (6) regions, and all the voltage measurements taken in each region may be averaged to be representative of the current response for the particular region.

With further reference to FIG. 9A, the method 900 at block 930 calculates intermediate analyte concentrations of the analyte. For example, numerous intermediate analyte concentrations may be calculated, optionally using numerous scaling factors. The calculations may use an equation of the form:

$$G = c + {\sum }_{i = 1}^{N} {\sum }_{j = 1}^{N} {a}_{ij} {x}_{i} {x}_{j}$$

where
   G is a calculated intermediate analyte concentration,
   N is a number of a subset of the measured current values,
   xᵢ, for i=1 to N, are the subset of the measured current values,
   aᵢⱼ is a predetermined matrix of coefficients, and
   c is a constant.

The specific details of the calculations of four (4) intermediate analyte concentrations are set forth with respect to FIGS. 8A-8E above. For example, the method 900 at block 940 determines different subsets of the measured current values and different scaling factors. The different intermediate concentrations have different accuracies in different ranges of analyte concentrations. As such, the method 900 at block 950 makes use of these different intermediate concentrations in order to determine a resultant analyte concentration. The method 900 at block 960 can then calculate a bias factor and check for and/or report errors. Alternatively, the method 900 at block 970 may annunciate or report the analyte concentration to a patient.

Turning next to FIG. 9B, further details of the method 900 at block 950 determining the resultant analyte concentration are provided. Initially, the method 900 at block 951 determines if the temperature of the physiological fluid is within a predetermined temperature range at which a particular algorithmic determination may be applicable. In one example, the temperature range may be between 17° C. and 28° C. In another example, the temperature range may be between 22° C. and 25° C. If the temperature of the fluid is not within the predetermined range, then a first subset and a first scaling factor are selected to calculate a first intermediate analyte concentration. For example, the method 900 may proceed to block 952, and the first intermediate analyte concentration may be calculated as G₁, as set forth with respect to FIG. 8B above. In such a case, G₁ has a first reasonable level of accuracy across a range of analyte concentrations ranging from a low range to a high range. In other words, G₁ may be invariant to glucose concentration level across a broad range, thus providing a good “rough estimate” of the glucose concentration. For example, G₁ may be generally applicable from less than 50 mg/dL to well over 200 mg/dL.

If the temperature of the fluid is within the predetermined range, the method 900 at blocks 954, 956, 958 may be programmed to select a different calculation, depending on the outcome of the G₁ calculation. For example, the method 900 at block 954 may select G₂, as set forth with respect to FIG. 8C above, if G₁ is indicative of a low glucose range, because the G₂ calculation may be more accurate in the low glucose range, such as a range less than 80 mg/dL. Similarly, the method 900 at block 956 may instead select G₃, as set forth with respect to FIG. 8D above, if G₁ is indicative of a high glucose range, because the G₃ calculation may be more accurate in the high glucose range, such as a range over 100 mg/dL. And in a case in which G₁ is indicative of a medium glucose range between the high and low ranges, such as between 80 and 100 mg/dL, the method 900 at block 958 may instead select the arithmetic average, or ½ (G₂+G₃). In another example, a weighted average (using weighting coefficients) or other average, such a geometric average, of G₂ and G₃ may be chosen.

Continuing with FIG. 9C, the method 900 at block 960 may calculate a fourth intermediate glucose concentration as an error check of the concentration determined by the method 900 at block 950. In one example, G₄, as set forth with respect to FIG. 8E above, may be chosen for performing the error check, which may be calculated as an absolute bias or a relative bias.

Initially, the method 900 at block 962 determines if the analyte concentration level is below a predetermined threshold, using, for example, the first intermediate analyte concentration G₁ to make the determination. If the analyte concentration level is not below the predetermined threshold, then the method 900 at block 964 checks if the absolute bias between G₁ and G₄ is below a predetermined threshold. For example, the predetermined threshold for absolute bias may be 25 mg/dL, 35 mg/dL, or another value between 10-50 mg/dL. If the analyte concentration is below the predetermined threshold, then the method 900 at block 966 checks if the relative bias between G₁ and G₄ is below a predetermined threshold. For example, the predetermined threshold for relative bias may be 40%, 35%, or another value between 10-50%. In either case, if the bias is not below the predetermined threshold, the method 900 at block 968 reports an error. Alternatively, if the bias is below the predetermined threshold, the method 900 at block 970 can report or annunciate the results of the calculation performed at block 950.

While the invention has been described in terms of particular variations and illustrative figures, those of ordinary skill in the art will recognize that the invention is not limited to the variations or figures described. In addition, where methods and steps described above indicate certain events occurring in certain order, those of ordinary skill in the art will recognize that the ordering of certain steps may be modified and that such modifications are in accordance with the variations of the invention. Additionally, certain of the steps may be performed concurrently in a parallel process when possible, as well as performed sequentially as described above. Therefore, to the extent there are variations of the invention, which are within the spirit of the disclosure or equivalent to the inventions found in the claims, it is the intent that this patent will cover those variations as well.

To the extent that the claims recite the phrase “at least one of” in reference to a plurality of elements, this is intended to mean at least one or more of the listed elements, and is not limited to at least one of each element. For example, “at least one of an element A, element B, and element C,” is intended to indicate element A alone, or element B alone, or element C alone, or any combination thereof. “At least one of element A, element B, and element C” is not intended to be limited to at least one of an element A, at least one of an element B, and at least one of an element C.

This written description uses examples to disclose the invention, including the best mode, and also to enable any person skilled in the art to practice the invention, including making and using any devices or systems and performing any incorporated methods. The patentable scope of the invention is defined by the claims, and may include other examples that occur to those skilled in the art. Such other examples are intended to be within the scope of the claims if they have structural elements that do not differ from the literal language of the claims, or if they include equivalent structural elements with insubstantial differences from the literal language of the claims.

The terminology used herein is for the purpose of describing particular embodiments only and is not intended to be limiting. As used herein, the singular forms “a,” “an,” and “the” are intended to include the plural forms as well, unless the context clearly indicates otherwise. It will be further understood that the terms “comprise” (and any form of comprise, such as “comprises” and “comprising”), “have” (and any form of have, such as “has” and “having”), “include” (and any form of include, such as “includes” and “including”), and “contain” (and any form of contain, such as “contains” and “containing”) are open-ended linking verbs. As a result, a method or device that “comprises,” “has,” “includes,” or “contains” one or more steps or elements possesses those one or more steps or elements, but is not limited to possessing only those one or more steps or elements. Likewise, a step of a method or an element of a device that “comprises,” “has,” “includes,” or “contains” one or more features possesses those one or more features, but is not limited to possessing only those one or more features. Furthermore, a device or structure that is configured in a certain way is configured in at least that way, but may also be configured in ways that are not listed.

The corresponding structures, materials, acts, and equivalents of all means or step plus function elements in the claims below, if any, are intended to include any structure, material, or act for performing the function in combination with other claimed elements as specifically claimed. The description set forth herein has been presented for purposes of illustration and description, but is not intended to be exhaustive or limited to the form disclosed. Many modifications and variations will be apparent to those of ordinary skill in the art without departing from the scope and spirit of the disclosure. The embodiment was chosen and described in order to best explain the principles of one or more aspects set forth herein and the practical application, and to enable others of ordinary skill in the art to understand one or more aspects as described herein for various embodiments with various modifications as are suited to the particular use contemplated.

What is claimed is:

1. A method for determining a concentration of an analyte in a physiological fluid with a biosensor having at least two electrodes, the method comprising:
applying at least three voltage pulses across the two electrodes, the at least three voltage pulses comprising at least two pulses of opposite polarity;
measuring current values at one or more of the two electrodes during each of the three voltage pulses;
calculating intermediate analyte concentrations of the analyte including a first intermediate analyte concentration using a first subset of the measured current values and a first scaling factor, a second intermediate analyte concentration using a second subset of the measured current values and a second scaling factor, and a third intermediate analyte concentration using a third subset of the measured current values and a third scaling factor, wherein
the first subset and the first scaling factor are selected to provide the calculated first intermediate analyte concentration with a first level of accuracy across a range of analyte concentrations ranging from a low range to a high range,
the second subset and the second scaling factor are selected to provide the calculated second intermediate analyte concentration with a second level of accuracy higher than the first level of accuracy in the low range of the analyte concentrations, and
the third subset and the third scaling factor are selected to provide the calculated third intermediate analyte concentration with a third level of accuracy higher than the first level of accuracy in the high range of the analyte concentrations; and
determining the concentration of the analyte as a function of the first, second and third intermediate analyte concentrations, the determining comprising
selecting the second intermediate analyte concentration responsive to the first intermediate analyte concentration being in the low range,
selecting the third intermediate analyte concentration responsive to the first intermediate analyte concentration being in the high range, and
selecting an average of the second and third intermediate analyte concentrations responsive to the first intermediate analyte concentration being between the low and the high ranges.

2. The method of claim 1, wherein calculating each of the intermediate analyte concentrations comprises using an equation of the form
$$G = c + {\sum }_{i = 1}^{N} {\sum }_{j = 1}^{N} {a}_{ij} {x}_{i} {x}_{j}$$
where
G is a calculated intermediate analyte concentration,
N is a number of a subset of the measured current values,
xᵢ, for i=1 to N, are the subset of the measured current values,
aᵢⱼ, for i=1 to N and j=1 to N, are predetermined coefficients, and
c is a constant.

3. The method of claim 1, wherein the determining further comprises selecting the first intermediate analyte concentration responsive to a temperature of the physiological fluid being outside a predetermined temperature range.

4. The method of claim 3, wherein the predetermined temperature range comprises between 17° C. and 28° C.

5. The method of claim 1, further comprising:
calculating a fourth intermediate analyte concentration of the analyte using at least one of the current values measured during the third voltage pulse without using a scaling factor;
calculating a relative bias value between the determined analyte concentration and the fourth intermediate analyte concentration; and
reporting an error responsive to the relative bias value being greater than a predetermined amount.

6. The method of claim 1, wherein the analyte comprises glucose, the low range comprises less than 80 mg/dL, and the high range comprises greater than 100 mg/dL.

7. The method of claim 6, further comprising:
calculating a fourth intermediate analyte concentration of the analyte using at least one of the current values measured during the third voltage pulse without using a scaling factor;
calculating an absolute bias value between the determined analyte concentration and the fourth intermediate analyte concentration; and
reporting an error responsive to the determined analyte concentration being less than 100 mg/dL and the absolute bias value being 25 mg/dL or greater.

8. The method of claim 1, wherein the at least three voltage pulses comprise a first positive voltage pulse having a duration of about 2 seconds, a second negative voltage pulse having a duration of about 1 second and a third positive voltage pulse having a duration of about 1.5 seconds.

9. The method of claim 8, wherein the at least three voltage pulses comprises a zero voltage pulse delay having a duration of about 0.5 seconds.

10. The method of claim 1, wherein the at least three voltage pulses comprise a first positive voltage pulse configured to measure a diffusion-limited reaction of the analyte and a reagent of the biosensor and a second negative voltage pulse configured to measure a kinetic-limited reaction of the analyte and the reagent.

11. The method of claim 1, wherein the at least two electrodes comprise a working electrode and a counter electrode, and the measuring

12. The method of claim 1, wherein each of the second subset and the third subset includes one or more of the current values measured during each of the at least three voltage pulses, and the second and third subsets are different subsets of the current values.

13. The method of claim 1, wherein the first, second and third scaling factors are different scaling factors.

14. The method of claim 1, wherein the first subset of the current values comprises all of the current values.

15. A method for determining a concentration of an analyte in a physiological fluid with a biosensor having at least two electrodes, the method comprising:
applying at least three voltage pulses across the two electrodes, the at least three voltage pulses comprising at least two pulses of opposite polarity;
measuring current values at one or more of the two electrodes during each of the three voltage pulses;
calculating intermediate analyte concentrations of the analyte including a first intermediate analyte concentration using a first subset of the measured current values and a first scaling factor, a second intermediate analyte concentration using a second subset of the measured current values and a second scaling factor, a third intermediate analyte concentration using a third subset of the measured current values and a third scaling factor, and a fourth intermediate analyte concentration of the analyte using at least one of the current values measured during the third voltage pulse without using a scaling factor, wherein
the first subset and the first scaling factor are selected to provide the calculated first intermediate analyte concentration with a first level of accuracy across a range of analyte concentrations ranging from a low range to a high range,
the second subset and the second scaling factor are selected to provide the calculated second intermediate analyte concentration with a second level of accuracy higher than the first level of accuracy in the low range of the analyte concentrations, and
the third subset and the third scaling factor are selected to provide the calculated third intermediate analyte concentration with a third level of accuracy higher than the first level of accuracy in the high range of the analyte concentrations;
determining the concentration of the analyte as a function of the first, second and third intermediate analyte concentrations, the determining comprising
selecting the first intermediate analyte concentration responsive to a temperature of the physiological fluid being outside a predetermined temperature range,
selecting the second intermediate analyte concentration responsive to the first intermediate analyte concentration being in the low range,
selecting the third intermediate analyte concentration responsive to the first intermediate analyte concentration being in the high range, and
selecting an average of the second and third intermediate analyte concentrations responsive to the first intermediate analyte concentration being between the low and the high ranges;
calculating a relative bias value between the determined analyte concentration and the fourth intermediate analyte concentration; and
reporting an error responsive to the relative bias value being greater than a predetermined amount.

16. The method of claim 15, wherein calculating each of the intermediate analyte concentrations comprises using an equation of the form
$$G = c + {\sum }_{i = 1}^{N} {\sum }_{j = 1}^{N} {a}_{ij} {x}_{i} {x}_{j}$$
where
G is a calculated intermediate analyte concentration,
N is a number of a subset of the measured current values,
xᵢ, for i=1 to N, are the subset of the measured current values,
aᵢⱼ is a predetermined matrix of coefficients, and
c is a constant.

17. The method of claim 15, wherein the predetermined temperature range comprises between 17° C. and 28° C.

18. The method of claim 15, wherein the at least three voltage pulses comprise a first positive voltage pulse having a duration of about 2 seconds, a second negative voltage pulse having a duration of about 1 second and a third positive voltage pulse having a duration of about 1.5 seconds.

19. A system for determining a concentration of an analyte in a physiological fluid, the system comprising:
a biosensor having at least two electrodes; and
a meter configured to
apply at least three voltage pulses across the two electrodes, the at least three voltage pulses comprising at least two pulses of opposite polarity,
measure current values at one or more of the two electrodes during each of the three voltage pulses,
calculate intermediate analyte concentrations of the analyte including a first intermediate analyte concentration using a first subset of the measured current values and a first scaling factor, a second intermediate analyte concentration using a second subset of the measured current values and a second scaling factor, and a third intermediate analyte concentration using a third subset of the measured current values and a third scaling factor, wherein
the first subset and the first scaling factor are selected to provide the calculated first intermediate analyte concentration with a first level of accuracy across a range of analyte concentrations ranging from a low range to a high range,
the second subset and the second scaling factor are selected to provide the calculated second intermediate analyte concentration with a second level of accuracy higher than the first level of accuracy in the low range of the analyte concentrations, and
the third subset and the third scaling factor are selected to provide the calculated third intermediate analyte concentration with a third level of accuracy higher than the first level of accuracy in the high range of the analyte concentrations, and
determine the concentration of the analyte as a function of the first, second and third intermediate analyte concentrations by
selecting the second intermediate analyte concentration responsive to the first intermediate analyte concentration being in the low range,
selecting the third intermediate analyte concentration responsive to the first intermediate analyte concentration being in the high range, and
selecting an average of the second and third intermediate analyte concentrations responsive to the first intermediate analyte concentration being between the low and the high ranges.

20. The system of claim 19, wherein the meter is configured to calculate each of the intermediate analyte concentrations comprises using an equation of the form
$$G = c + {\sum }_{i = 1}^{N} {\sum }_{j = 1}^{N} {a}_{ij} {x}_{i} {x}_{j}$$
where
G is a calculated intermediate analyte concentration,
N is a number of a subset of the measured current values,
xᵢ, for i=1 to N, are the subset of the measured current values,
aᵢⱼ is a predetermined matrix of coefficients, and
c is a constant.
